(19) United States
(12) Patent Application Publication
(10) Pub. No.: US 2009/0028809 A1
(43) Pub. Date: Jan. 29, 2009

(54) Personal care article for sequentially dispensing compositions with variable concentrations of hydrophobic benefit materials

(76) Inventors: Jonathan Robert Cetti, Mason, OH (US); Christopher Dean Putman, West Chester, OH (US); Karen Lehnhoff, Cincinnati, OH (US); Tommy Lee Williams, JR., West Chester, OH (US); Edward Dewey Smith, III, Mason, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 11/881,551

(22) Filed: Jul. 27, 2007

(51) Int. Cl. A61K 8/00

(52) U.S. Cl. 424/70.1

(57) ABSTRACT

The present invention relates to a personal care article for providing at least two liquid personal care compositions. The personal care article comprises a single chamber package and a liquid personal care product. The package comprises a dispensing orifice, a first zone proximate, to the dispensing orifice and a second zone distal to the dispensing orifice. The liquid personal care product comprises a first personal care composition substantially disposed within the first zone and the second personal care composition substantially disposed within the second zone. The first personal care composition comprises a first concentration of hydrophobic benefit material. The second personal care composition comprises a second concentration of hydrophobic benefit material. The first concentration of hydrophobic benefit material is different from the second concentration of hydrophobic benefit material.

### FIELD OF THE INVENTION

The present invention relates to a personal care article that provides a liquid personal care product that comprises at least two compositions each having a concentration of hydrophobic benefit material which is noticeably distinct from each other.

### BACKGROUND OF THE INVENTION

Personal care compositions are well known and widely used for cleansing and moisturizing skin and hair, delivering actives, hiding imperfections, to reducing the oiliness/shine, as well as, providing scent to the shower and/or the skin. The efficacy of these types of compositions is directly related to their frequency of use and level of active ingredients. In some cases, a high level of benefit agent in a personal care composition will maintain a benefit to a consumer for several days after a single application. In this case, a full bottle of the composition with a high level of benefit agent is not needed because the continued application of personal care composition with high level of benefit agent would not provide additional benefit to the consumer over one or two single applications. Numerous cosmetic applications require that the corresponding compositions be used at variable dose of active ingredients in the course of time. Up until now, it order to carry out these treatments, the available resources have consisted either of successive applications of increasing or decreasing active ingredient percentages in separate containers or multiplying the applications of compositions with identical active ingredients percentages in order to obtain the correct does for the necessary treatment. If a treatment regime contains too many steps or too many containers, consumers often habituate or tire of the regime of personal care compositions over time. When this habituation occurs consumers often decrease or even or stop use of one personal care product despite the benefits gained by the compliant use of the regime of personal care products over time. With the space in the shower or bath being limited, a typical shower or bath does not have enough space, to place multiple containers of personal care compositions so that a consumer can easily switch the use of one personal care composition to another personal care composition with a different level of benefit agent.

### SUMMARY OF THE INVENTION

The present invention relates to a personal care article for providing at least two liquid personal care compositions. The personal care article comprises a single chamber package and a liquid personal care product. The package comprises a dispensing orifice, a first zone proximate to the dispensing orifice and a second zone distal to the dispensing orifice. The liquid personal care product comprises a first personal care composition substantially disposed within the first zone and the second personal care composition substantially disposed within the second zone. The first personal care composition comprises a first concentration of hydrophobic benefit material. The second personal care composition comprises a second concentration of hydrophobic benefit material. The first concentration of hydrophobic benefit material is different from the second concentration of hydrophobic benefit material. Thus, the personal care article of the present invention comprises a liquid personal care product that changes in moisturization level as it is dispensed from the package which overcomes the problem of a regime that involves too many steps or too many containers.

### BRIEF DESCRIPTION OF THE DRAWINGS

FIGS. 1A and 1B illustrate a personal care article with three zones having horizontal interfaces between the compositions in each zone.

### DETAILED DESCRIPTION OF THE INVENTION

The term “ambient conditions” as used herein, refers to surrounding conditions at one (1) atmosphere of pressure, 50% relative humidity, and 25° C.

As used herein, “comprising” means that other steps and other ingredients which do not affect the end result can be added. This term encompasses the terms “consisting of” and “consisting essentially of”. The compositions and methods/processes of the present invention can comprise, consist of, or consist essentially of the essential elements and limitations of the invention described herein, as well as any of the additional or optional ingredients, components, steps, or limitations described herein useful in personal cleansing compositions intended for topical application to the hair or skin.

The term “liquid” as used herein means that the composition is generally flowable to some degree. “Liquids”, therefore, may include liquid, semi-liquid, cream, lotion or gel compositions intended for topical application to skin. The compositions may exhibit a viscosity of equal to or greater than about 1,500 (centipoise, hereinafter “cps”), equal to or greater than about 5,000 cps, equal to or greater than about 10,000 cps or equal to or greater than about 20,000 cps and no more than about 1,000,000 cps, no more than about 500,000 cps, no more than about 300,000 cps, or no more than about 200,000 cps as measured by the T-Bar Viscosity Method described hereinafter.

The term “package” includes any suitable container for personal care compositions exhibiting a viscosity from about 1,500 centipoise (cP) to about 1,000,000 cP, including but not limited to a bottle, tottle, tube, jar, non-aerosol pump and mixtures thereof. As used herein “tottle” refers to a bottle which rests on the neck or mouth which its contents are filled in and dispensed from, but it is also the end upon which the bottle is intended to rest or sit upon for storage by the consumer and/or for display on the store shelf, as described in the commonly owned U.S. patent application Ser. No. 11/067443 filed on Feb. 25, 2005 to McCall, et al, entitled “Multi-phase Personal Care Compositions, Process for Making and Providing, and Article of Commerce.”

The term “personal care composition” as used herein, refers to compositions intended for topical application to the skin or hair. The compositions of the present invention are rinse-off formulations, in which the product is applied topically to the skin or hair and then is subsequently rinsed within minutes from the skin or hair with water, or otherwise wiped off using a substrate with deposition of a portion of the composition. The compositions also may be used as shaving aids. The personal care composition of the present invention is typically extrudable or dispensable from a single chamber package. The personal care compositions of the present invention can be in the form of liquid, semi-liquid, cream, lotion or gel compositions intended for topical application to skin. Examples of personal care compositions of the present invention can include but are not limited to shampoo, conditioning shampoo, hair conditioner, body wash, moisturizing body wash, shower gels, skin cleansers, cleansing milks, hair and body wash, in shower body moisturizer, pet shampoo, shaving preparations and cleansing compositions used in conjunction with or applied to a disposable cleansing cloth. The personal care compositions of the present invention are typically in the form of a liquid. The product forms contemplated for purposes of defining the compositions and methods of the present invention are rinse-off formulations by which it is meant that the product is applied topically to the skin or hair and then subsequently (i.e., within minutes) rinsed away with water, or otherwise wiped off using a substrate or other suitable removal means.

The term “stable” as used herein, unless otherwise specified, refers to a personal care product that comprise at least two compositions that maintain at least two “separate” zones with at least two separate benefit concentrations zones contained within a single chamber package at ambient conditions for a period of at least about 180 days. By “separate” is meant that there is substantially no mixing of compositions contained in said zones, detected by the benefit analysis method, described hereinafter, prior to dispensing of the composition.

The term “structured,” as used herein means having a rheology that confers stability on the personal care composition. The degree of structure is determined by characteristics determined by one or more of the following methods the Yield Stress Method, or the Zero Shear Viscosity Method or by the Ultracentrifugation Method, all in the Test Methods below. Accordingly, a surfactant phase of the composition of the present invention is considered “structured,” if the surfactant phase has one or more of the following properties described below according to the Yield Stress Method, or the Zero Shear Viscosity Method or by the Ultracentrifugation Method. A surfactant phase is considered to be structured, if the phase has one or more of the following characteristics:
A. a Yield Stress of greater than about 0.1 Pascal (Pa), more preferably greater than about 0.5 Pa, even more preferably greater than about 1.0 Pa, still more preferably greater than about 2.0 Pa, still even more preferably greater than about 3 Pa, and even still even more preferably greater than about 5 Pa as measured by the Yield Stress and Zero Shear Viscosity Method described hereafter:
B. a Zero Shear Viscosity of at least about 500 Pascal-seconds (Pa-s), preferably at least about 1,000 Pa-s, more preferably at least about 1,500 Pa-s, even more preferably at least about 2,000 Pa-s; or
C. a Structured Domain Volume Ratio as measured by the Ultracentrifugation Method described hereafter, of greater than about 40%, preferably at least about 45%, more preferably at least about 50%, more preferably at least about 55%, more preferably at least about 60%, more preferably at least about 65%, more preferably at least about 70%, more preferably at least about 75%, more preferably at least about 80%, even more preferably at least about 85%.

The term “surfactant component” as used herein means the total of all anionic, nonionic, amphoteric, zwitterionic and cationic surfactants in a phase. When calculations are based on the surfactant component, water and electrolyte are excluded from the calculations involving the surfactant component, since surfactants as manufactured typically are diluted and neutralized.

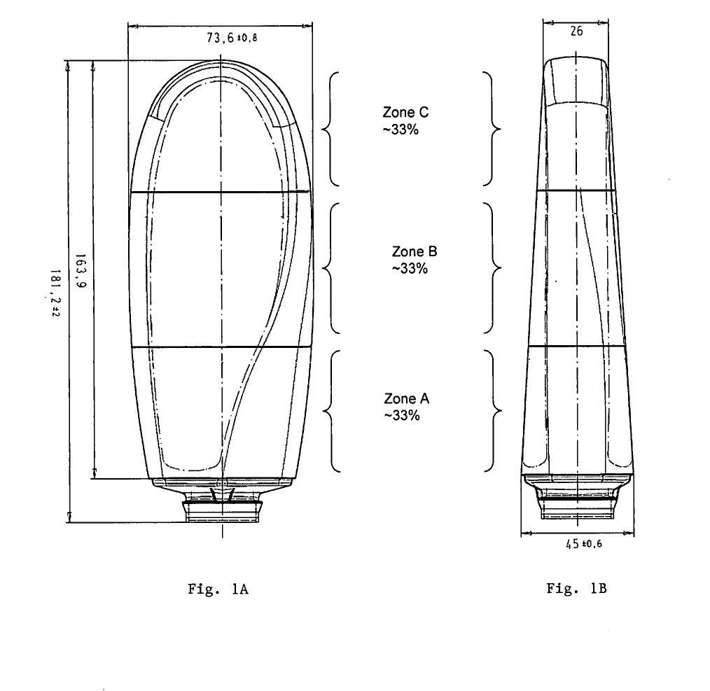

As used herein the term “zone” is a domain or region within a single chamber package which corresponds to a composition of the personal care product. An interface between the zones can be distinct or gradual or separated by another zone. The amount contained within a zone can be defined by a percentage of the package volume and a zone comprises at least 10% of the package volume of a given package, excluding the volume of the package corresponding to the closure, as shown in FIGS. 1A and 1B of the present invention.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein. Except where specific examples of actual measured values are presented, numerical values referred to herein should be considered to be qualified by the word “about”.

All molecular weights as used herein are weight average molecular weights expressed as grams/mole, unless otherwise specified.

The present invention relates to a personal care article that provides single chamber package comprising a liquid personal care product. The liquid personal care product comprises at least two personal care compositions, each composition having a noticeably distinct hydrophobic benefit material concentration. These distinct concentrations can be dispensed sequentially from the package. For example, a package could dispense a high level of hydrophobic benefit material, followed by a composition with a medium level of hydrophobic benefit material, followed by a composition with a lower level of hydrophobic benefit material concentration. Thus, the liquid personal care product changes in moisturization level as it is dispensed from the package which overcomes the problem of a regime that involves too many steps or too many containers.

The present invention relates to a personal care article for providing at least two liquid personal care compositions. The personal care article comprises a single chamber package and a liquid personal care product. The package comprises a dispensing orifice, a first zone proximate to the dispensing orifice and a second zone distal to the dispensing orifice. The liquid personal care product comprises a first personal care composition substantially disposed within the first zone and the second personal care composition substantially disposed within the second zone. In one aspect, the first zone is in physical contact with the second zone within the single chamber package. In one aspect, the first personal care composition is in physical contact with the second personal care composition within the single chamber package

The personal care article for dispensing and or applying at least two liquid personal care compositions comprises a single chamber package that comprises at least two zones with at least two personal care compositions substantially disposed within the respective zones. The number of zones with a package and thus, the number of personal care compositions disposed within the respective zone can vary in number. For example, the package may have three zones and three personal care composition within the respective zones; four zones and four compositions, five zones and five compositions, and so on. In one aspect, the personal care article comprises a third zone medial to the dispensing orifice. In one aspect, the personal care article comprising a third personal care composition substantially disposed within the third zone; the third personal care composition comprising a third concentration of a hydrophobic benefit material wherein the third concentration is different from the first concentration and the second concentration. In another aspect, the first zone, the second zone and the third zone comprise an equal percentage, by volume, of the package.

In another aspect, each personal care composition may comprise a dye, colorant or the like, such that each personal care composition is a distinct color or hue. For example, the first personal care composition can be a yellow color, the second personal care composition can be a orange color and the third personal care composition can be a purple color.

The amount of hydrophobic benefit materials in compositions are usually formulated, by weight of the composition, at less than about 55%, less than about 45%, less than about 30%, less than about 20%, less than about 10%, less than about 5%, less than about 4%, less than about 3%, less than about 2%, less than about 1%. Each personal care composition may comprise from about 1.0% to about 60%, from about 5% to about 60%, from about 10% to about 50%, from about 20% to about 45%, by weight of the personal care composition, of a hydrophobic benefit material. In one aspect of the personal care article of the present invention, the first personal care composition or the second composition of the present invention may comprise a concentration of 0% hydrophobic benefit material.

The compositions of the present invention can be multi-phase and comprise one of more phases or one or more of the components described in the phases below:

The personal care composition of the present invention can comprise a cleansing phase comprising components of the cleansing phase. The personal care composition typically comprises from about 1% to about 100%, by weight of the composition; from about 5% to about 85%; by weight of the composition, from about 10% to 80%, by weight of the composition; from about 20 to 70%, by weight of the composition; from about 25% to 60%, by weight of the composition, from about 30% to about 50%, by weight of the composition, of a cleansing phase.

The cleansing phase can comprise a structured domain that is comprised of a mixture of surfactants. The presence of structured domain enables the incorporation of high levels of hydrophobic benefit materials in a separate phase which is not emulsified within composition. In one aspect, the structured domain in the composition can be characterized as, or is, an opaque structured domain. In one aspect, the opaque structured domain can be characterized as, or is, a lamellar phase. The lamellar phase produces a lamellar gel network. The lamellar phase can provide resistance to shear, adequate yield to suspend particles and droplets and at the same time provides long term stability, since it is thermodynamically stable. The lamellar phase tends to have a higher viscosity thus minimizing the need for viscosity modifiers.

In one aspect, cleansing phase can comprise a domain that is comprised of a mixture of surfactants and can be a micellar phase. A micellar phase is optically isotropic. Micelles are approximately spherical in shape. Other shapes such as ellipsoids, cylinders, and bilayers are also possible. In one aspect, the micellar phase can be structured to enhance viscosity and to suspend particles. This can be accomplished using viscosity modifiers such as those defined below as water structurants.

The cleansing phase comprises a surfactant component which can be comprised of a mixture of surfactants including lathering surfactants or a mixture of lathering surfactants. The cleansing phase comprises surfactants suitable for application to the mammalian skin or hair and are compatible with water and the other ingredients of the composition of the present invention. These surfactants include anionic, nonionic, cationic, zwitterionic, amphoteric, soap, or combinations thereof. Preferably, anionic surfactant comprises at least 40% of the surfactant component. The personal care composition can comprise the surfactant component at concentrations ranging from about 2% to about 40%, from about 4% to about 25%, about 1% to about 21%, about 3 to 15%, by weight of the composition, of the surfactant component.

Suitable surfactants are described in McCutcheon's, Detergents and Emulsifiers, North American edition (1986), published by allured Publishing Corporation; and McCutcheon's, Functional Materials, North American Edition (1992); and in U.S. Pat. No. 3,929,678 issued to Laughlin, et al on Dec. 30, 1975.

Preferred linear anionic surfactants for use in the structured surfactant phase of the personal care composition include ammonium lauryl sulfate, ammonium laureth sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, potassium lauryl sulfate, and combinations thereof.

Branched anionic surfactants and monomethyl branched anionic surfactants suitable for the present invention are described in a commonly owned, patent application published on December 2006 under U.S. Publication No. 60/680,149 entitled “Structured Multi-phased Personal Cleansing Compositions Comprising Branched Anionic Surfactants” filed on May 12, 2005 by Smith, et al. Branched anionic surfactants include but are not limited to the following surfactants: sodium trideceth sulfate, sodium tridecyl sulfate, sodium C₁₂₋₁₃ alkyl sulfate, and C₁₂₋₁₃ pareth sulfate and sodium C₁₂₋₁₃ pareth-n sulfate.

In one aspect of the personal care compositions of the present invention may further preferably comprise an amphoteric surfactant, a zwitterionic surfactant and mixtures thereof. In one embodiment, the personal care composition can comprise at least one amphoteric surfactant. Amphoteric surfactant suitable for use in the present invention include those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products described in U.S. Pat. No. 2,528,378. In one aspect, the personal care composition can comprise an amphoteric surfactant that is selected from the group consisting of sodium lauroamphoacetate, sodium cocoamphoactetate, disodium lauroamphoacetate disodium cocodiamphoacetate, and mixtures thereof. Moreover, Amphoacetates and diamphoacetates can also be used.

Zwitterionic surfactants suitable for use include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Zwitterionic surfactants suitable for use in the personal care composition include alkyl betaines, including cocoamidopropyl betaine.

The personal care composition of the present invention is preferably free of alkyl amines and alkanolamide to ensure mildness of the composition to the skin.

An electrolyte can be added per se to the personal care composition or it can be formed in situ via the counterions included in one of the raw materials. The electrolyte preferably includes an anion comprising phosphate, chloride, sulfate or citrate and a cation comprising sodium, ammonium, potassium, magnesium or mixtures thereof. Some preferred electrolytes are sodium chloride, ammonium chloride, sodium or ammonium sulfate. The electrolyte is preferably added to the structured surfactant phase of the composition in the amount of from about 0.1% to about 6%; from about 1% to about 5%, more preferably from about 2% to about 4%, more preferably from about 3% to about 4%, by weight of the personal care composition.

The first personal care composition can comprise a first concentration of surfactant and second personal care composition can comprise a second concentration of surfactant. The first concentration of surfactant can be different from the second concentration of surfactant. In one aspect, the first personal care composition can a first concentration of surfactant that is a greater that the second concentration of surfactant in the second personal care compositions. In one aspect, the first personal care composition can have a lower concentration of surfactant than the second personal care compositions.

The personal care compositions of the present invention comprise a benefit phase or benefit phase components. The benefit phase in the present invention is preferably anhydrous and can be substantially free of water. The benefit phase can be substantially free or free of surfactant.

Hydrophobic benefit materials suitable for use in the present invention preferably have a Vaughan Solubility Parameter of from about 5 (cal/cm³)^{1/2} to about 15 (cal/cm³)^{1/2}, as defined by Vaughan in Cosmetics and Toiletries, Vol. 103. The Vaughan Solubility Parameter (VSP) as used herein is a parameter used to define the solubility of hydrophobic materials. Vaughan Solubility parameters are well known in the various chemical and formulation arts and typically have a range of from 5 to 25. Non-limiting examples of hydrophobic benefit materials having VSP values ranging from about 5 to about 15 include the following: Cyclomethicone 5.92, Squalene 6.03, Petrolatum 7.33, Isopropyl Palmitate 7.78, Isopropyl Myristate 8.02, Castor Oil 8.90, Cholesterol 9.55, as reported in Solubility, Effects in Product, Package, Penetration and Preservation, C. D. Vaughan, Cosmetics and Toiletries, Vol. 103, October 1988.

The hydrophobic benefit materials for use in the benefit phase of the composition have a preferred rheology profile as defined by Consistency value (k) and Shear Index (n). The term “Consistency value” or “k” as used herein is a measure of lipid viscosity and is used in combination with Shear Index, to define viscosity for materials whose viscosity is a function of shear. The measurements are made at 35° C. and the units are poise (equal to 100 cps). The term “Shear Index” or “n” as used herein is a measure of lipid viscosity and is used in combination with Consistency value, to define viscosity for materials whose viscosity is a function of shear. The measurements are made at 35° C. and the units are dimensionless. Consistency value (k) and Shear Index (n) are more fully described in the Test Methods below. Preferred Consistency value ranges are 1-10,000 poise (1/sec)ⁿ⁻¹, preferably 10-2000 poise (1/sec)ⁿ⁻¹ and more preferably 50-1000 poise (1/sec)ⁿ⁻¹. Shear Index ranges are 0.1-0.8, preferably 0.1-0.5 and more preferably 0.20-0.4. These preferred rheological properties are especially useful in providing the personal cleansing compositions with improved deposition of benefit agents on skin.

The benefit phase can be comprised of the hydrophobic benefit materials selected from the group consisting of petrolatum, lanolin, derivatives of lanolin (e.g. lanolin oil, isopropyl lanolate, acetylated lanolin, acetylated lanolin alcohols, lanolin alcohol linoleate, lanolin alcohol riconoleate) hydrocarbon oils (e.g. mineral oil) natural and synthetic waxes (e.g. micro-crystalline waxes, paraffins, ozokerite, lanolin wax, lanolin alcohols, lanolin fatty acids, polyethylene, polybutene, polydecene, pentahydrosqualene) volatile or non-volatile organosiloxanes and their derivatives (e.g. dimethicones, cyclomethicones, alkyl siloxanes, polymethylsiloxanes, methylphenylpolysiloxanes), natural and synthetic triglycerides (e.g. castor oil, soy bean oil, sunflower seed oil, maleated soy bean oil, safflower oil, cotton seed oil, corn oil, walnut oil, peanut oil, olive oil, cod liver oil, almond oil, avocado oil, palm oil, sesame oil) and combinations thereof. In one aspect, at least about 50% by weight of the hydrophobic benefit materials are selected from the groups of petrolatum, mineral oil, paraffins, polyethylene, polybutene, polydecene, dimethicones, alkyl siloxanes, cyclomethicones, lanolin, lanolin oil, lanolin wax. The remainder of the hydrophobic benefit material can be selected from: isopropyl palmitate, cetyl riconoleate, octyl isononanoate, octyl palmitate, isocetyl stearate, hydroxylated milk glyceride and combinations thereof. The benefit phase of the personal care composition can be comprised a combination of petrolatum and mineral oil.

The personal care compositions of the present invention can comprise a structured aqueous phase which can comprise a water structurant and water. The structured aqueous phase can be hydrophilic. In one aspect, the structured aqueous phase can be a hydrophilic, non-lathering gelled water phase. The structured aqueous phase can comprises less than about 5%; less than about 3%; less than about 1%, by weight of the structured aqueous phase, of a surfactant component. In one apect, the structured aqueous phase can be is free of lathering surfactants in the composition. The structured aqueous phase of the present invention can comprise from about 30% to about 99%, more than about 50%, more than about 60%, more than about 70%, more than about 80%, by weight of the structured aqueous phase, of water.

The structured aqueous phase can comprise in some aspects a water structurant. The water structurant is selected from the group consisting of inorganic water structurants (e.g. silicas, polyacrylates, polyacrylamides, modified starches, crosslinked polymeric gellants, copolymers) charged polymeric water structurants (e.g. Acrylates/Vinyl Isodecanoate Crosspolymer (Stabylen 30 from 3V), Acrylates/C10-30 Alkyl Acrylate Crosspolymer (Pemulen TR1 and TR2), Carbomers, Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant), Acrylates/Ceteth-20 Itaconate Copolymer (Structure 3001 from National Starch), Polyacrylamide (Sepigel 305 from SEPPIC), water soluble polymeric structurants (e.g. cellulose gums and gel, and starches), associative water structurants (e.g. xanthum gum, gellum gum, pectins, alginates such as propylene glycol alginate), and mixtures thereof. The structured aqueous phase can comprise from about 0.1% to about 30%, from about 0.5% to about 20%, from about 0.5% to about 10%, and from about 0.5% to about 5%, by weight of the structured aqueous phase, of a water structurant. A water structurant for the structured aqueous phase can have a net cationic charge, net anionic charge, or neutral charge.

The structured aqueous phase can have a pH in the range from about 5 to about 9.5, or in one aspect have a pH of about 7. The structured aqueous phase of the present compositions can further comprise optional ingredients such as, pigments, pH regulators (e.g. triethanolamine), and preservatives.

While not essential for the purposes of the present invention, the non-limiting list of optional materials, illustrated hereinafter are suitable for use in personal care compositions, and may be incorporated in certain embodiments, for example to assist or enhance cleansing performance, for treatment of the skin, or to modify the aesthetics of the personal care composition. Optional materials useful in the products herein are described by their cosmetic and/or therapeutic benefit or their postulated mode of action or function. These descriptions are non-limiting and made for the sake of convenience because it is understood that these materials can provide more than one benefit, function or operate via more than one mode of action. The precise nature of these optional materials, and levels of incorporation thereof, will depend on the physical form of the composition and the nature of the cleansing operation for which it is to be used. The amount of optional materials in compositions are usually formulated, by weight of the composition, at less than about less than about 6%, less than about 5%, less than about 4%, less than about 3%, less than about 2%, less than about 1%, less than about 0.5%, less than about 0.25%, less than about 0.1%, less than about 0.01%, less than about 0.005%.

Optional ingredients, which can be used in the personal care compositions of the present invention, can be selected from the group consisting of thickening agents; low density microspheres (e.g. Expancel 091 WE40 d24, Akzo Nobel and others described in commonly owned and assigned U.S. Patent Publication No. 2004/0092415A1 published on May 13, 2004); preservatives; antimicrobials; fragrances; chelators (e.g. such as those described in U.S. Pat. No. 5,487,884 issued to Bisset, et al.); sequestrants; vitamins (e.g. Retinol); vitamin derivatives (e.g. tocophenyl actetate, niacinamide, panthenol); sunscreens; desquamation actives (e.g. such as those described in U.S. Pat. Nos. 5,681,852 and 5,652,228 issued to Bisset); anti-wrinkle/anti-atrophy actives (e.g. N-acetyl derivatives, thiols, hydroxyl acids, phenol); anti-oxidants (e.g. ascorbic acid derivatives, tocophenol) skin soothing agents/skin healing agents (e.g. panthenoic acid derivatives, aloe vera, allantoin); skin lightening agents (e.g. kojic acid, arbutin, ascorbic acid derivatives) skin tanning agents (e.g. dihydroxyacteone); polymeric phase structurant (e.g. naturally derived polymers, synthetic polymers, crosslinked polymers, block copolymers, copolymers, hydrophilic polymers, nonionic polymers, anionic polymers, hydrophobic polymers, hydrophobically modified polymers, associative polymers, and oligomers); a liquid crystalline phase inducing structurant (e.g. trihydroxystearin available from Rheox, Inc. under the trade name THIXCIN® R); organic cationic deposition polymer (e.g. Polyquatemium 10 available from Amerchol Corp. Edison, N.J., USA, guar hydroxypropyltrimonium chloride available as Jaguar C-17 from Rhodia Inc., and N-Hance polymer series commercially available from Aqualon); pH regulators (e.g. triethanolamine); anti-acne medicaments; essential oils; sensates; pigments; colorants; pearlescent agents; interference pigments (e.g such as those disclosed in U.S. Pat. No. 6,395,691 issued to Liang Sheng Tsaur, U.S. Pat. No. 6,645,511 issued to Aronson, et al., U.S. Pat. No. 6,759,376 issued to Zhang, et al, U.S. Pat. No. 6,780,826 issued to Zhang, et al.) particles (e.g. talc, kolin, mica, smectite clay, cellulose powder, polysiloxane, silicas, carbonates, titanium dioxide, polyethylene beads) hydrophobically modified non-platelet particles (e.g. hydrophobically modified titanium dioxide and other materials described in a commonly owned, patent application published on Aug. 17, 2006 under Publication No. 2006/0182699A by Taylor, et al.) and mixtures thereof. Other optional ingredients are most typically those materials approved for use in cosmetics and that are described in the CTFA Cosmetic Ingredient Handbook, Second Edition, The Cosmetic, Toiletries, and Fragrance Association, Inc. 1988, 1992.

### Test Methods

### Benefit Analysis Method:

This method determines the weight ratio of cleansing (surfactant) phase to lipid phase in dual phase composition. A sample of dual-phase composition is mixed and tested using a moisture analyzer for % moisture. The result is calculated by dividing the total % moisture in the composition by the % moisture in the surfactant phase then multiplying that result by 100. The % benefit agent (lipid) is calculated by subtracting the % surfactant phase from 100. It is applicable only to dual phase compositions in which one phase (lipd) contributes no volatiles at the temperature conditions used in the instrument program.

Apparatus:

| | |
|---|---|
| Infared or Halogen | Programed according to the operating manual, |
| Moisture Balance (e.g. | using the following test parameters: |
| Mettler-Toledo HR73 | Heating mode: Ramp to 140° C. in 5 minutes |
| Moisture Analyzer) | Switchoff mode: 3 |
| aluminum drying pans | Aluminum drying pans must be dried and |
| (4 inch × 5/16 inch deep) | stored in a desiccator prior to use. Dry the |
| | pans in a conventional oven for 1 hour at |
| | 130° C. Allow pans to cool to room |
| | temperature before using. Always handle pans |
| | with a clean pair of forceps |
| 1 ml Disposable Syringes | |
| Analytical Balance | capable of weighing to 3 decimal places |

### T-Bar Viscosity Method:

The viscosity of a composition contained within a zone can be assessed by the T-Bar Viscosity Method. In the case of testing from a product package, two zones can be selected from the package that contains at least two compositions that contain separate hydrophobic benefit material concentrations. In order to separate the zones, the product can be frozen at a temperature of at least −20° C. for a period of at least 24 hours. The zones are then cut using a cutting implement such as a bandsaw. The cut portions are collected separately and allowed equilibrate to ambient conditions.

The apparatus for T-Bar measurement includes a Brookfield DV-II+ Pro Viscometer with Helipath Accessory; chuck, weight and closer assembly for T-bar attachment; a T-bar Spindle D, a personal computer with Rheocalc software from Brookfield, and a cable connecting the Brookfield Viscometer to the computer. First, weigh 80 grams of the first or second composition in a 4-oz glass jar. Measure the T-bar viscosity by carefully dropping the T-Bar Spindle to the interior bottom of the jar and set the Helipath stand to travel in an upward direction. Open the Rheocalc software and set the following data acquisition parameters: set Speed to 5 rpm, set Time Wait for Torque to 00:01 (1 second), set Loop Start Count at 100. Start data acquisition and turn on the Helipath stand to travel upward at a speed of 22 mm/min. The T-Bar viscosity “T” is the average T-Bar viscosity reading between the 6^{th} reading and the 95^{th} reading (the first five and the last five readings are not used for the average T-Bar viscosity calculation). If the viscosity is below the lower limit of the D spindle (30,000 cps), a larger spindle can be used for the T-Bar Viscosity measurement.

### Ultracentrifugation Method:

The Ultracentrifugation Method is used to determine the percent of a structured domain or an opaque structured domain that is present in a multi-phase personal care composition that comprises a structured surfactant phase comprising a surfactant component. The method involves the separation of the composition by ultracentrifugation into separate but distinguishable layers. The multi-phase personal care composition of the present invention can have multiple distinguishable layers, for example a non-structured surfactant layer, a structured surfactant layer, and a benefit layer.

First, dispense about 4 grams of multi-phase personal care composition into Beckman Centrifuge Tube (11×60 mm). Next, place the centrifuge tubes in an Ultracentrifuge (Beckman Model L8-M or equivalent) and ultracentrifuge using the following conditions: 50,000 rpm, 18 hours, and 25° C.

After ultracentrifuging for 18 hours, determine the relative phase volume by measuring the height of each layer visually using an Electronic Digital Caliper (within 0.Olmm). First, the total height is measured as Hₐ which includes all materials in the ultracentrifuge tube. Second, the height of the benefit layer is measured as H_{b}. Third, the structured surfactant layer is measured as H_{c}. The benefit layer is determined by its low moisture content (less than 10% water as measured by Karl Fischer Titration). It generally presents at the top of the centrifuge tube. The total surfactant layer height (Hₛ) can be calculated by this equation:

Hₛ=Hₐ−H_{b}

The structured surfactant layer components may comprise several layers or a single layer. Upon ultracentrifugation, there is generally an isotropic layer at the bottom or next to the bottom of the ultracentrifuge tube. This clear isotropic layer typically represents the non-structured micellar surfactant layer. The layers above the isotropic phase generally comprise higher surfactant concentration with higher ordered structures (such as liquid crystals). These structured layers are sometimes opaque to naked eyes, or translucent, or clear. There is generally a distinct phase boundary between the structured layer and the non-structured isotropic layer. The physical nature of the structured surfactant layers can be determined through microscopy under polarized light. The structured surfactant layers typically exhibit distinctive texture under polarized light. Another method for characterizing the structured surfactant layer is to use X-ray diffraction technique. Structured surfactant layer display multiple lines that are often associated primarily with the long spacings of the liquid crystal structure. There may be several structured layers present, so that H_{c} is the sum of the individual structured layers. If a coacervate phase or any type of polymer-surfactant phase is present, it is considered a structured phase.

Finally, the structured domain volume ratio is calculated as follows:

Structured Domain Volume Ratio=H_{c}/Hₛ*100%

If there is no benefit phase present, use the total height as the surfactant layer height, Hₛ=Hₐ.

### Yield Stress and Zero Shear Viscosity Method:

The Yield Stress and Zero Shear Viscosity of a composition contained within a zone, can be measured either prior to combining the phases in a composition, or after combining the phases in a composition by separating the phases by suitable physical separation means, such as centrifugation, pipetting, cutting away mechanically, rinsing, filtering, or other separation means. In the case of testing from a product package, two zones can be selected from the package that contains at least two compositions that contain separate hydrophobic benefit material concentrations. In order to separate the zones, the product can be frozen at a temperature of at least −20° C. for a period of at least 24 hours. The zones are then cut using a cutting implement such as a bandsaw. The cut portions are collected separately and allowed equilibrate to ambient conditions.

A controlled stress rheometer such as a TA Instruments AR2000 Rheometer is used to determine the Yield Stress and Zero Shear Viscosity. The determination is performed at 25° C. with the 4 cm diameter parallel plate measuring system and a 1 mm gap. The geometry has a shear stress factor of 79580 m⁻³ to convert torque obtained to stress. Serrated plates can be used to obtain consistent results when slip occurs.

First a sample of the composition is obtained and placed in position on the rheometer base plate, the measurement geometry (upper plate) moving into position 1 mm above the base plate. Excess phase at the geometry edge is removed by scraping after locking the geometry. If the phase comprises particles discernible to the eye or by feel (beads, e.g.) which are larger than about 150 microns in number average diameter, the gap setting between the base plate and upper plate is increased to the smaller of 4 mm or 8-fold the diameter of the 95^{th} volume percentile particle diameter. If a phase has any particle larger than 5 mm in any dimension, the particles are removed prior to the measurement.

The determination is performed via the programmed application of a continuous shear stress ramp from 0.1 Pa to 1,000 Pa over a time interval of 4 minutes using a logarithmic progression, i.e., measurement points evenly spaced on a logarithmic scale. Thirty (30) measurement points per decade of stress increase are obtained. Stress, strain and viscosity are recorded. If the measurement result is incomplete, for example if material flows from the gap, results obtained are evaluated and incomplete data points excluded. The Yield Stress is determined as follows. Stress (Pa) and strain (unitless) data are transformed by taking their logarithms (base 10). Log(stress) is graphed vs. log(strain) for only the data obtained between a stress of 0.2 Pa and 2.0 Pa, about 30 points. If the viscosity at a stress of 1 Pa is less than 500 Pa-sec but greater than 75 Pa-sec, then log(stress) is graphed vs. log(strain) for only the data between 0.2 Pa and 1.0 Pa, and the following mathematical procedure is followed. If the viscosity at a stress of 1 Pa is less than 75 Pa-sec, the zero shear viscosity is the median of the 4 highest viscosity values (i.e., individual points) obtained in the test, the yield stress is zero, and the following mathematical procedure is not used. The mathematical procedure is as follows. A straight line least squares regression is performed on the results using the logarithmically transformed data in the indicated stress region, an equation being obtained of the form:

Log(strain)=m*Log(stress)+b (1)

Using the regression obtained, for each stress value (i.e., individual point) in the determination between 0.1 and 1,000 Pa, a predicted value of log(strain) is obtained using the coefficients m and b obtained, and the actual stress, using Equation (1). From the predicted log(strain), a predicted strain at each stress is obtained by taking the antilog (i.e., 10^{x} for each x). The predicted strain is compared to the actual strain at each measurement point to obtain a % variation at each point, using Equation (2).

% variation=100*(measured strain−predicted strain)/measured strain (2)

The Yield Stress is the first stress (Pa) at which % variation exceeds 10% and subsequent (higher) stresses result in even greater variation than 10% due to the onset of flow or deformation of the structure. The Zero Shear Viscosity is obtained by taking a first median value of viscosity in Pascal-seconds (Pa-sec) for viscosity data obtained between and including 0.1 Pa and the Yield Stress. After taking the first median viscosity, all viscosity values greater than 5-fold the first median value and less than 0.2× the median value are excluded, and a second median viscosity value is obtained of the same viscosity data, excluding the indicated data points. The second median viscosity so obtained is the Zero Shear Viscosity.

### EXAMPLES

### Examples

The following example described in Table 1 shows non-limiting examples of the articles containing multi-phase composition with variant level of hydrophobic benefit materials throughout the bottle of the present invention and a comparative example that does not have a variant level of hydrophobic benefit materials. Refer to FIGS. 1A and 1B which defines the zones described below.

TABLE 1
Examples of the Present Invention and Comparative Example

| Examples of the Present | | |
|---|---|---|
| Invention | | Comparative |
| A | B | Example |

Structured Surfactant Phase Composition

| | | | |
|---|---|---|---|
| Sodium Lauroamphoacetate | 4.9 | 4.9 | 4.9 |
| (Cognis Chemical Corp.,) | | | |
| Sodium Trideceth Sulfate | 8.4 | 8.4 | 8.4 |
| (sulfated from Iconol TDA-3 (BASF Corp.) to | | | |
| >95% sulfate) | | | |
| Sodium Lauryl Sulfate | 8.4 | 8.4 | 8.4 |
| Trideceth-3 | 2.0 | 2.0 | 2.0 |
| (Iconal TDA-3 from BASF Corp.) | | | |
| Sodium Chloride | 4.75 | 4.75 | 4.75 |
| Guar hydroxypropyltrimonium chloride | 0.6 | 0.6 | 0.6 |
| (N-Hance 3196 Polymer) | | | |
| Polyethyleneoxide | 0.15 | 0.15 | 0.15 |
| (Polyox WSR301) | | | |
| Xanthan gum (Keltrol 1000, Kelco Corp.) | 0.2 | 0.2 | 0.2 |
| Hollow microspheres (Expancel 091 WE40 | 0.36 | 0.3 | 0.3 |
| d24, Akzo Nobel) | | | |
| Methyl chloro isothiazolinone and methyl | 0.0005 | 0.0005 | 0.0005 |
| isothiazolinone (Kathon CG, Rohm & Haas) | | | |
| EDTA (Dissolvine NA 2x) | 0.15 | 0.15 | 0.15 |
| Sodium Benzoate | 0.2 | 0.2 | 0.2 |
| Citric Acid, titrate | pH = 5.7 ± 0.2 | pH = 5.7 ± 0.2 | pH = 5.7 ± 0.2 |
| Perfume | 1.3 | 1.3 | 1.3 |
| Water | Q.S. | Q.S. | Q.S. |

Benefit Phase Composition

| | | | |
|---|---|---|---|
| Petrolatum | 70 | 70 | 70 |
| (from Quidesa, Mexico) | | | |
| Hydrobrite 1000 White Mineral Oil (from | 30 | 30 | 30 |
| WITCO, USA) | | | |
| Cosmetic Pigment, Red 7 Ca Lake | 0.01 | 0.01 | 0.01 |
| Surfactant Phase to Benefit Phase Ratio (by | 65:35 | 55:45 | 55:45 |
| weight): Zone A | | | |
| Surfactant Phase to Benefit Phase Ratio (by | 50:50 | 65:35 | 55:45 |
| weight): Zone B | | | |
| Surfactant Phase to Benefit Phase Ratio (by | 60:40 | 75:25 | 55:45 |
| weight): Zone C | | | |

The compositions described above can be prepared by conventional formulation and mixing techniques. Prepare the structured surfactant phase composition by first adding citric acid into water at 1:3 ratios to form a citric acid premix. Prepare a polymer premix by adding Polyox WSR301 and Xanthan Gum into Trideceth-3 (Example A) or Isosteareth-2 (Example B and Comparative Example). Then, add the following ingredients into the main mixing vessel in the following sequence with agitation: water, N-Hance polymer, Expancel, sodium lauroamphoacetate, sodium trideceth sulfate, sodium sodium lauroamphoacetate, sodium lauryl sulfate, sodium chloride, sodium benzoate, and Disodium EDTA. Add citric acid premix to adjust pH to 5.7±0.2. Add the polymer premix into the main mixing vessel with continuous agitation. Add perfume while continuing to agitate until homogeneous.

Prepare the benefit phase composition by first adding petrolatum into a mixing vessel. Heat the vessel to 180° F. (82.2° C.). Then, add Hydrobrite 1000 White mineral oil and cosmetic pigment (Example A) with agitation. Let the vessel cool down with slow agitation to about 110° F. (43.3° C.) and transfer the lipid to a container to cool down to ambient overnight.

A visually distinct multiphase composition of the present invention can be prepared by melting the benefit phase and combining at a specified ratio with a surfactant phase of the present invention in a transparent package while the package is rotated. A multiphase composition of the present invention can also be prepared by optionally melting the benefit phase and combining with a surfactant phase of the present invention in an agitated tank or using agitation from a static mixer to create a dispersion of one phase in the other, then filling the composition into a package.

### Filling the Empty Product Container to Form a Container of Multiphase Composition

The multiphase personal care composition can be prepared by any suitable means. For example, filling the empty product container with the multiphase personal care composition can comprise transferring predetermined amounts of the different phases through at least one dispensing means into an empty product container. Additionally, the multiphase personal care compositions can be prepared by the method and apparatus as disclosed in U.S. Pat. No. 6,213,166 issued to Thibiant, et al. on Apr. 10, 2001. The method and apparatus allows two or more compositions to be filled with a spiral configuration into a single container. The method requires that at least two nozzles be employed to fill the container. The container is placed on a moving stage and spun as the composition is introduced into the container.

Alternatively, it is effective to combine at least two phases by first placing the separate compositions in separate storage tanks having a pump and a hose attached. The phases are then pumped in predetermined amounts into a single combining section. Next, the phases are moved from the combining sections into a blending section and the phases are mixed in the blending section. The next step involves pumping the product that was mixed in the blending section via a hose into a single nozzle, then placing the nozzle into a container and filing the container with the resulting product such that the single resulting product exhibits a visually distinct non-random pattern of the phases.

The shape and size of the particular product container used will dictate the rate of filling, rotation of the bottle, frequency of rotation of the bottle, and movement of the bottle during filling, as these factors can have a direct impact on the shape, size, and overall appearance of the pattern in the multiphase composition. The starting position of the bottle (proximity to the multiphase dispenser, as well as relative position when initially being filled), also affect the appearance of the pattern in the multiphase composition.

Example A and Example C from Table 1 were analyzed according to the benefit analysis method. Shown in Table 2 and 3 are the results.
% Lipid Intended represents the intended % Lipid by weight in the composition.
% Lipid Dispensed: 10 g samples were dispensed from the tottle in a 20 ml vial, and mixed with a spatula until blended. Sample numbers 1, 12, and 25 were analyzed according to the benefit analysis method representing Zone A (1^{st} dispensing), Zone B (12^{th} dispensing) and Zone C (25^{th} dispensing) of the tottle as defined in FIGS. 1A and 1B.
% Lipid Sectioned: The tottle filled with the composition was frozen at −29 oC for 24 hours. The three zones as defined in FIGS. 1A and 1B were cut using a bandsaw, and the compositions contained within the cut portions were collected in a 8 oz. jar and allowed to equilibrate to ambient conditions. Once the samples were equilibrated, they were mixed with a spatula until blended. The portions were then analyzed according to the benefit analysis method.

TABLE 2
Example A from Table 1 - % Lipid Results
of a composition with varying benefit agent

| | % Lipid | % Lipid | % Lipid |
|---|---|---|---|
| Bottle Position | Intended | Sectioned | Dispensed |
| Zone A (Containing 1st | 35.00 | 43.64 | 37.80 |
| dispensing) | | | |
| Zone B (Containing 12th | 50.00 | 49.61 | 50.80 |
| dispensing) | | | |
| Zone C (Containing 25th | 40.00 | 39.23 | 40.01 |
| dispensing) | | | |

TABLE 3
Example C from Table 1 - % Lipid Results of a comparative
composition with the same level of benefit agent throughout

| | % Lipid | % Lipid | % Lipid |
|---|---|---|---|
| Bottle Position | Intended | Sectioned | Dispensed |
| Zone A (Containing 1st | 50.00 | 51.08 | 52.70 |
| dispensing) | | | |
| Zone B (Containing 12th | 50.00 | 53.06 | 53.78 |
| dispensing) | | | |
| Zone C (Containing 25th | 50.00 | 55.42 | 53.65 |
| dispensing) | | | |

The inventors were able to conclude from the data provided in table 2 and table 3 that a bottle can be filled with a variant level of hydrophobic benefit materials. Furthermore, the inventors were able to conclude from the data provided in table 2 and table 3, that the product dispensed can deliver approximately the same level of hydrophobic benefit materials as the actual level sectioned from the different zones of the bottle.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) A personal care article for dispensing and or applying a liquid personal care product comprising:
a) a single chamber package comprising a dispensing orifice, a first zone proximate to said dispensing orifice and a second zone distal to said dispensing orifice; and
b) a liquid personal care product comprises a first personal care composition substantially disposed within the first zone and a second personal care composition substantially disposed within the second zone;
wherein said first composition comprises a first concentration of a hydrophobic benefit material;
wherein said second composition comprises a second concentration of a hydrophobic benefit material; and
wherein said first concentration is different from said second concentration.

2) The personal care article of claim 1 wherein said first concentration of said hydrophobic benefit agent is greater than said second concentration of said hydrophobic benefit agent.

3) The personal care article of claim 1 wherein said second concentration of said hydrophobic benefit agent is greater than said first concentration of said hydrophobic benefit agent.

4) The personal care article of claim 1 wherein said first concentration of said hydrophobic benefit agent is zero.

5) The personal care article of claim 1 wherein said second concentration of said hydrophobic benefit agent is zero.

6) The personal care article of claim 1 wherein said first zone comprises from about 10% to about 90%, by volume, of said package.

7) The personal care article of claim 1 wherein said first zone comprises from about 30% to about 70%, by volume of said package.

8) The personal care article of claim 1 wherein said first zone comprises about 50%, by volume of said package.

9) The personal care article of claim 1, wherein said first personal care composition comprises a first concentration of surfactant and said second personal care composition comprises a second concentration of surfactant.

10) The personal care article of claim 1, wherein said first concentration of surfactant is greater than said second concentration of surfactant.

11) The personal care article of claim 1, wherein said first personal care composition comprises a lamellar phase.

12) The personal care article of claim 1, wherein said second personal care composition comprises a lamellar phase.

13) The personal care article of claim 1 wherein said first zone is in physical contact with said second zone within said package.

14) The personal care article of claim 1 wherein said first personal care composition is a distinct hue from said second personal care composition.

15) The personal care article of claim 1 further comprising a third zone medial to said dispensing orifice.

16) The personal care article of claim 15 further comprising a third personal care composition substantially disposed within said third zone; said third personal care composition comprising a third concentration of a hydrophobic benefit material wherein said third concentration is different from said first concentration and said second concentration.

17) The personal care article of claim 1 wherein said first zone, said second zone and said third zone comprise an equal percentage, by volume, of said package.

18) The personal care article of claim 1 wherein said first composition further comprising polythethylene beads.

19) The personal care article of claim 17 wherein said second composition further comprising titanium dioxide.

20) The personal care article of claim 17 wherein said second composition further comprising interference pigment.
